(19) United States
(12) Patent Application Publication
(10) Pub. No.: US 2022/0370048 A1
(43) Pub. Date: Nov. 24, 2022

(54) PORTABLE OROPHARYNGEAL SWAB SMART COLLECTION DEVICE

(71) Applicant: SOUTHERN UNIVERSITY OF SCIENCE AND TECHNOLOGY, Shenzhen, Guangdong (CN)

(72) Inventors: Hongqiang WANG, Shenzhen (CN); Ting WANG, Shenzhen (CN); Renjie ZHU, Shenzhen (CN); Dongliang FAN, Shenzhen (CN); Yifan ZHANG, Shenzhen (CN); Guojie XU, Shenzhen (CN); Hao LIU, Shenzhen (CN)

(73) Assignee: SOUTHERN UNIVERSITY OF SCIENCE AND TECHNOLOGY, Shenzhen, Guangdong (CN)

(21) Appl. No.: 17/626,650

(22) PCT Filed: Apr. 23, 2021
(86) PCT No.: PCT/CN2021/089106

§ 371 Date: Jan. 12, 2022

(30) Foreign Application Priority Data
Jul. 27, 2020 (CN) 202010731589.8

(51) Int. Cl. A61B 10/00; A61B 5/00

(52) U.S. Cl. CPC A61B 10/0051; A61B 5/742; A61B 5/746; A61B 2090/064

(57) ABSTRACT

A portable oropharyngeal swab smart collection device, which includes a collection device body, a remote control module, and a visual display unit, where a main control unit and an execution unit and a visual unit which are electrically connected with the main control unit respectively are arranged on the collection device body; a detachable oropharyngeal swab collector is arranged on the execution unit; the remote control module includes remote operation equipment which is in communication connection with the main control unit; and the visual display unit is in communication connection with the main control unit and the visual unit. The portable oropharyngeal swab smart collection device of the present disclosure can implement a collection by a oropharyngeal swab through remote control, rather than a face-to-face artificial collection by the oropharyngeal swab by a medical staff to a suspected patient with an infectious disease.

### FIELD OF THE APPLICATION

The present disclosure relates to the field of medical apparatus and instruments, in particular to a portable oropharyngeal swab smart collection device.

### BACKGROUND

At present, there are three clinical sampling modes: oropharyngeal sampling, nasal cavity sampling, and deep sputum sampling, which are operated manually. A medical staff collects a specimen at a pathological site of a patient by relevant medical apparatus and instruments, and submits the specimen for examination to fulfill an objective of pathological diagnosis.

A pathological sampling at an oropharyngeal position, that is, a sampling by a oropharyngeal swab, is the most common pathological sampling mode clinically. At present, in most cases, the sampling is operated manually, that is, the medical staff puts a sampling bar of the oropharyngeal swab into a mouth of the patient for sampling. As there is a possibility in a face-to-face contact between the medical staff and the patient, a sampling staff is extremely easy to infect, and even though there are no cough and sneezing for the patient, there is still a possibility of an infection through droplets. A sampling site can be accessible to the pathological site preferably (where there is a common swelling or pyosis); and if there is no any obvious pathological tissue, the sampling sites (which are mostly palatine tonsil, pharyngopalatine arch, and posterior pharyngeal wall) are scrubbed by a sampling apparatus both vertically and horizontally or in a round motion.

However, the current artificial sampling by the oropharyngeal swab has the following defects:

1. During the sampling at the oropharyngeal position, generally, any site where there is the swelling or the pyosis is the strongest pathologically, and thus, a specimen at the site is high in positive rate. However, at present, the sampling staff judges the sampling site artificially, on one hand , it is difficult to judge whether or not there is the swelling or the pyosis accurately, while on the other hand, part of sampling staff don't examine whether or not there are the swelling, the pyosis, and other pathological sites, or may have a psychological repellence during the sampling due to a vomiting of the patient; and the operation staff may take the specimens quickly at as many sites as possible, which leads to reduction in a sampling efficiency and a sampling accuracy.

2. During the artificial sampling, the sampling staff completely relies on a subjective feeling in controlling a sampling effort, which may easily increase a painful feeling of a sampling object due to the improper sampling effort, and even cause an injury to a biological tissue of a human body.

3. During the artificial sampling, the mouth of the patient is directly exposed to the sampling staff, and even though there is no cough or sneezing for the patient, there are still the droplets spilled out of the patient, making the sampling staff encounter a great infection risk.

Therefore, the prior art needs to be improved and developed.

### BRIEF SUMMARY OF THE DISCLOSURE

In view of defects of the prior art, the present disclosure aims to provide a portable oropharyngeal swab smart collection device to solve problems during an artificial sampling by a oropharyngeal swab, such as reduction in a sampling efficiency and a sampling accuracy, and easiness in infection of an operation staff, in the prior art.

A technical solution of the present disclosure is as follow:

The portable oropharyngeal swab smart collection device includes a collection device body, a remote control module, and a visual display unit, where a main control unit and an execution unit and a visual unit which are electrically connected with the main control unit respectively are arranged on the collection device body; a detachable oropharyngeal swab collector is arranged on the execution unit; the remote control module includes remote operation equipment which is in communication connection with the main control unit; and the visual display unit is in communication connection with the main control unit and the visual unit.

According to the portable oropharyngeal swab smart collection device, where the main control unit is used for receiving an operation instruction sent by the remote operation equipment for controlling the execution unit to run.

According to the portable oropharyngeal swab smart collection device, where the execution unit includes a linear motion sub-unit which is arranged on the collection device body, a tail end execution sub-unit which is arranged on the linear motion sub-unit, and a position control sub-unit which is electrically connected with the main control unit.

According to the portable oropharyngeal swab smart collection device, where the tail end execution sub-unit includes a first brushless DC (Direct Current) motor which is horizontally installed on the linear motion sub-unit, and a second brushless DC motor which is vertically installed on the first brushless DC motor; and the first brushless DC motor and the second brushless DC motor are electrically connected with the main control unit.

According to the portable oropharyngeal swab smart collection device, where a sensing unit for acquiring a corresponding physical parameter is also arranged on the collection device body; the sensing unit is in communication connection with the visual display unit; the visual display unit is used for displaying the physical parameter transmitted by the sensing unit; and the sensing unit is electrically connected with the main control unit.

According to the portable oropharyngeal swab smart collection device, where a first emergency stop unit which is electrically connected with the main control unit is also arranged on the collection device body; and the remote control module further includes a second emergency stop unit which is in communication connection with the main control unit.

According to the portable oropharyngeal swab smart collection device, where a detachable disposable bite block unit and an expansion interface with which external equipment is connected are arranged on the collection device body.

According to the portable oropharyngeal swab smart collection device, where an alarm unit is also arranged on the collection device body; and the alarm unit is in communication connection with the main control unit.

According to the portable oropharyngeal swab smart collection device, where a remote voice unit is further arranged, which is used for a communication exchange between an operator and a patient.

Beneficial effect: compared with the prior art, the portable oropharyngeal swab smart collection device provided by the present disclosure has the following advantages:

1) the device is small in overall volume, portable, and low in cost, which can be popularized in outpatient services or communities;

2) the device is compact in appearance, approachable, and free of strong industrial sense like common medical apparatus and instruments, which can relieve a fear and a psychological repellence of the patient greatly;

3) the device is extremely convenient to transport, store, and disinfect, and there is no any dead angle of disinfection during disinfection, which can reduce a risk of a cross infection, and improve a safety coefficient;

4) the device is simple in structure and assembly, which is conductive to mass production, and can be applied in a front-line medical treatment quickly;

5) when the operator implements a remote control to the device as described by the present disclosure to collect a oropharyngeal swab specimen of the patient, there is no direct contact between the operator and the patient, which reduces the risk of an infection for a medical staff (operator) greatly;

6) compared with an ordinary artificial sampling, a standard ability of a sampling process is improved, the obtained specimen is high in quality, and a probability of examination missing and false negative is reduced;

7) the collection device body of the present disclosure is separated from the patient, and is equipped with a disposable bite block unit, which is replaced after being used by the patient at a time, thereby reducing the risk of the cross infection between the patients;

8) the collection device body of the present disclosure is controlled in a sampling effort during operation, which reduces a risk of an injury to a patient tissue greatly, and reduces a pain degree of the patient; and

9) the expansion interface is reserved on the collection device body of the present disclosure, to which multiple carriers can be connected externally according to needs.

### BRIEF DESCRIPTION OF THE DRAWINGS

FIG. 1 is a structural schematic diagram illustrating a portable oropharyngeal swab smart collection device of the present disclosure.

FIG. 2 is a connection schematic diagram illustrating an internal circuit of a portable oropharyngeal swab smart collection device of the present disclosure.

FIG. 3 is a structural schematic diagram illustrating a collection device body of the present disclosure.

### DETAILED DESCRIPTION

The present disclosure provides a portable oropharyngeal swab smart collection device. In order to understand the objectives, technical solutions, and effects of the present disclosure clearly, the present disclosure will be explained in details. It should be understood that the embodiments described herein are only illustrative of the present disclosure, and are not construed as a limitation to the present disclosure.

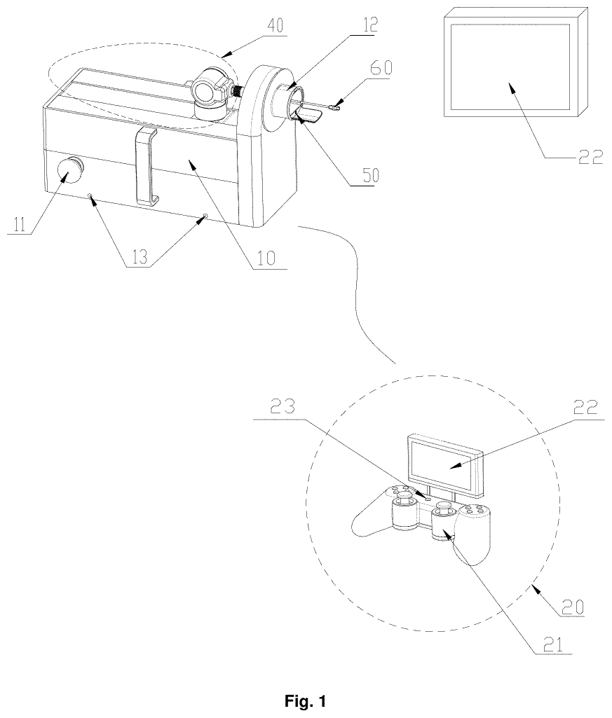

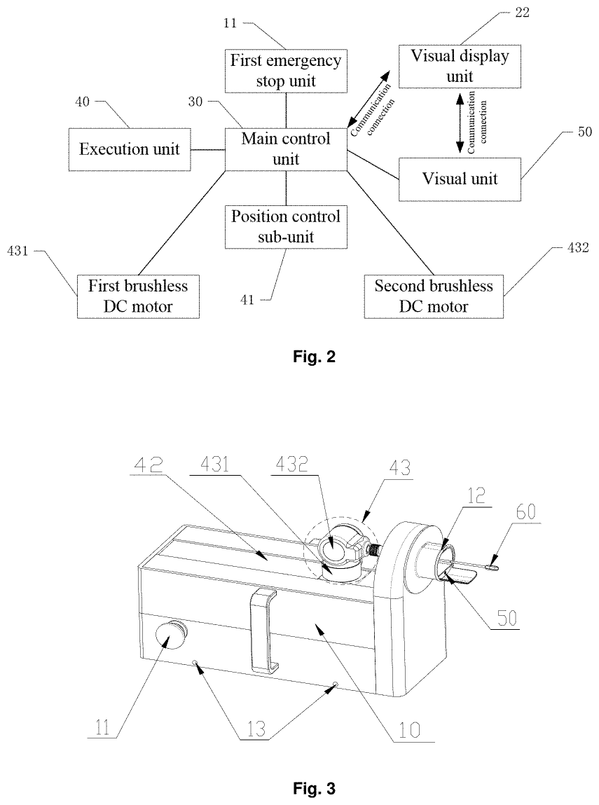

By referring to FIGS. 1 and 2, the present disclosure provides a portable oropharyngeal swab smart collection device, as shown in the figures, it includes a collection device body 10, a remote control module 20, and a visual display unit 22, where a main control unit 30 and an execution unit 40 and a visual unit 50 which are electrically connected with the main control unit 30 respectively are arranged on the collection device body 10; a detachable oropharyngeal swab collector 60 is arranged on the execution unit 40; the remote control module 20 includes remote operation equipment 21 which is in communication connection with the main control unit 30; the visual display unit 22 is in communication connection with the main control unit 30 and the visual unit 50; and the main control unit 30 is used for receiving an operation instruction sent by the remote operation equipment 21 for controlling the execution unit 40 to run.

Particularly, an operation process of the portable oropharyngeal swab smart collection device is as follow: the main control unit 30 receives the operation instruction sent by the remote operation equipment 21; the main control unit 30 controls the execution unit 40 to run according to the operation instruction; the execution unit 40 drives the oropharyngeal swab collector 60 to take a specimen at a fixed position in an oral cavity of a patient until a specimen collection by a oropharyngeal swab is completed; and during the specimen collection by the oropharyngeal swab, the visual unit 50 can transmit a motion of the oropharyngeal swab collector 60 at an oropharyngeal site of the patient and a picture at the oropharyngeal site of the patient to the visual display unit 22 in real time, so that an operator can complete the specimen collection by the oropharyngeal swab accurately and efficiently by observing the picture.

The portable oropharyngeal swab smart collection device of the embodiment can implement the collection by the oropharyngeal swab through remote control, rather than a face-to-face artificial collection by the oropharyngeal swab by a medical staff to the suspected patient with an infectious disease, which can reduce a risk of an infection of the medical staff to a great extent. The portable oropharyngeal swab smart collection device has characteristics of small and portable equipment, and low cost, is suitable to be popularized in outpatient services or communities, and is conductive to relieving a fear and a psychological repellence of the patient to medical apparatus and instruments; and meanwhile, the equipment is easy to operate, which is conductive to an accurate operation of the medical staff, thereby improving a one-time success rate of the specimen collection.

In some implementations, the oropharyngeal swab collector 60 can be a cotton swab, and other structures for taking the oropharyngeal swab specimen.

In some implementations, the portable oropharyngeal swab smart collection device provided by the embodiment not only can be subjected to an artificial remote control by the remote control module 20, but can also be operated automatically by selecting a fixed operational program after setting different operational programs in the main control unit 30 to complete the smart collection by the oropharyngeal swab.

In some implementations, as shown in the FIGS. 1 to 3, the execution unit 40 includes a position control sub-unit 41 which is electrically connected with the main control unit, a linear motion sub-unit 42 which is arranged on the collection device body 10, and a tail end execution sub-unit 43 which is arranged on the linear motion sub-unit 42. The tail end execution sub-unit 43 includes a first brushless DC motor 431 which is horizontally installed on the linear motion sub-unit 42 and a second brushless DC motor 432 which is vertically installed on the first brushless DC motor 431; a rotating shaft of the first brushless DC motor 431 and a rotating shaft of the second brushless DC motor 432 are staggered spatially in an angle of 90°; and the first brushless DC motor 431 and the second brushless DC motor 432 are electrically connected with the main control unit 30. In the embodiment, the linear motion sub-unit 42, as a motion track of the first brushless DC motor 431 in a horizontal direction, is arranged on the collection device body 10; the second brushless DC motor and the first brushless DC motor constitute a two-shaft pan-tilt device, which can make motion in a two-dimensional plane; and the first brushless DC motor 431 and the second brushless DC motor 432 are electrically connected with the position control sub-unit 41.

In some implementations, the oropharyngeal swab collector 60 is fixedly arranged on the second brushless DC motor 432; and the main control unit 30 sends the instruction and controls the first brushless DC motor 431 and the second brushless DC motor 432 to make motion in the two-dimensional plane, thereby driving the oropharyngeal swab collector 60 to move at the oral cavity of the patient and collect the oropharyngeal swab specimen.

In some implementations, as shown in the FIGS. 1 and 2, a sensing unit is also arranged on the collection device body 10; and the sensing unit is used for acquiring a corresponding physical parameter.

Particularly, the sensing unit sends the corresponding physical parameter to the main control unit 30; and the main control unit 30 feeds back the corresponding physical parameter to the visual display unit 22. As an example, the sensing unit may be a force sensor; and the operator can control an acting force during the sampling of the oropharyngeal swab collector 60 in the oral cavity of the patient by observing a feedback result corresponding to a moment value directly on the visual display unit 22 to avoid an injury to the patient during the sampling.

In some implementations, an alarm unit is also arranged on the collection device body 10; and the alarm unit is in communication connection with the main control unit 30. In the embodiment, if the moment value received by the main control unit 30 is larger than a preset threshold, alarm information is sent to the alarm unit, and the alarm unit gives an alarm to remind the operator, thereby further avoiding the injury to the patient during the sampling.

In some implementations, the visual display unit 22 can notify the motion of the oropharyngeal swab collector 60 at an oropharyngeal site of the patient, a picture at the oropharyngeal site of the patient, the physical parameter fed back by the sensing unit, and a current running state and an instruction for a next operation of the collection device body to the patient and the operator respectively to achieve a human-computer interaction.

In some implementations, the visual display unit 22 can be separately arranged on one end of the collection device body, at which the patient can observe the motion of the oropharyngeal swab collector 60 at the oropharyngeal site and the picture at the oropharyngeal site directly, and can also see the physical parameter fed back by the sensing unit and the current running state and the instruction for the next operation of the collection device body by the visual display unit 22 to achieve the human-computer interaction.

In some implementations, the visual display unit 22 can be separately arranged on one end of the remote control module 20, at which the operator can observe the motion of the oropharyngeal swab collector 60 at the oropharyngeal site of the patient and the picture at the oropharyngeal site of the patient directly, and can also see the physical parameter fed back by the sensing unit, and the current running state and the instruction for the next operation of the collection device body by the visual display unit 22 to achieve the human-computer interaction.

In some implementations, as shown in FIG. 1, the visual display unit 22 can be arranged at both one end of the collection device body 10 and one end of the remote control module 20, at which the patient and the operator can see the motion of the oropharyngeal swab collector 60 at the oropharyngeal site and the picture at the oropharyngeal site, and can also see the physical parameter fed back by the sensing unit, and the current running state and the instruction for a next operation of the collection device body to achieve the better human-computer interaction.

In an implementation, the visual display unit 22 is a high-definition display screen; and the high-definition display screen keeps communication connection with the visual unit 50 and the main control unit 30.

In some implementations, as shown in the FIGS. 1 and 2, a first emergency stop unit 11 which is electrically connected with the main control unit 30 is also arranged on the collection device body 10; and the remote control module 20 further includes a second emergency stop unit 23 which is in communication connection with the main control unit 30.

Particularly, the first emergency stop unit 11 and the second emergency stop unit 23 in the embodiment are both emergency stop switches, which can directly cut off a power supply of the portable oropharyngeal swab smart collection device. The first emergency stop unit 11 is arranged on the collection device body 10 and is electrically connected with the main control unit; and if the first emergency stop unit 11 is triggered, the execution unit 40 can stop functioning imminently, and at the same time, information is sent to the visual display unit 22 via wired or wireless communication, so that the operator is notified at first time for a next processing. If the second emergency stop unit 23 is triggered, the main control unit 30 is notified via the wired or wireless communication, so that the execution unit 40 stops functioning imminently. It can be known from the above description that in order to ensure a safety of the collection device, a dual fail-safe mechanism is used in the embodiment, that is, the first emergency stop unit 11 is arranged on the collection device body 10, and the second emergency stop unit 23 is arranged on the remote control module 20; and in case of any emergency circumstance, the patient and the operator can cut off the power supply of the device, thereby improving a safety coefficient greatly while achieving a maximum guarantee on a safety of the patient.

In some implementations, as shown in the FIGS. 1 and 3, a detachable disposable bite block unit 12 is arranged on the collection device body 10. The disposable bite block unit 12 can be assembled and disassembled quickly. In principle, one patient uses one corresponding bite block unit; and prior to the sampling, the bite block unit is installed on the collection device body 10 by the patient or the medical staff; and upon the sampling, the bite block unit is discarded to prevent a cross infection, thereby improving the safety coefficient during the sampling. The disposable bite block unit 12 achieves an effect of keeping the patient in a mouth opening state; and its unique shape design is conductive to a complete exposure of a oropharyngeal site of the patient, and a relative position between the patient and the collection device body 10 can be fixed, which can improve a sampling efficiency and a success rate of the one-time sampling greatly.

In some implementations, as shown in the FIGS. 1 and 3, an expansion interface 13 is also arranged on the collection device body 10. In the embodiment, the portable oropharyngeal swab smart collection device can be externally connected to a plurality of carriers by the expansion interface 13. As an example, the portable oropharyngeal swab smart collection device can be externally connected to a movable trolley to achieve an automated movement of the device; and it can also be externally connected to a mechanical arm to adjust a spatial position and a posture of the device, thereby adapting to the patients with different heights and bodily forms. With the arrangement of the expansion interface 13, the application field of the portable oropharyngeal swab smart collection device is expanded greatly.

In some implementations, the visual unit 50 is a high-definition visual module; and the high-definition visual module is used for acquiring the picture at the oropharyngeal site of the patient. In one implementation, the high-definition visual module can be used as an endoscope.

In some implementations, a remote voice unit is further arranged in the portable oropharyngeal swab smart collection device, which is used for a communication exchange between the operator and the patient. As an example, when needing to give voice prompt or guidance, the operator can send a voice to the patient by the remote voice unit.

In view of the above, the portable oropharyngeal swab smart collection device provided by the prevent disclosure can implement the collection by the oropharyngeal swab through remote control, rather than the face-to-face artificial collection by the oropharyngeal swab by the medical staff to the suspected patient with the infectious disease, which can reduce the risk of the infection of the medical staff to a great extent. The portable oropharyngeal swab smart collection device has characteristics of small and portable equipment, and low cost, is suitable to be popularized in the outpatient services or the communities, and is conductive to relieving the fear and the psychological repellence of the patient to the medical apparatus and instruments; and meanwhile, the equipment is easy to operate, which is conductive to the accurate operation of the medical staff, thereby improving the one-time success rate of the specimen collection.

It should be understood that the applications of the present disclosure are not limited to the above examples. All improvements and changes made by an ordinary skilled in the art according to the above descriptions shall fall within the scope of protection of claims of the present disclosure.

1-10. (canceled)

11. A portable oropharygneal swab smart collection device, comprising: a collection device body, a remote control module, and a visual display unit, wherein a main control unit and an execution unit and a visual unit which are electrically connected with the main control unit respectively are arranged on the collection device body; a detachable oropharyngeal swab collector is arranged on the execution unit; the remote control module comprises a remote operation equipment which is in communication connection with the main control unit; and the visual display unit is in communication connection with the main control unit and the visual unit.

12. The portable oropharyngeal swab smart collection device according to claim 11, wherein the main control unit is used for receiving an operation instruction sent by the remote operation equipment for controlling the execution unit to run.

13. The portable oropharyngeal swab smart collection device according to claim 11, wherein the execution unit comprises a linear motion sub-unit which is arranged on the collection device body, a tail end execution sub-unit which is arranged on the linear motion sub-unit, and a position control sub-unit which is electrically connected with the main control unit.

14. The portable oropharyngeal swab smart collection device according to claim 13, wherein the tail end execution sub-unit comprises a first brushless DC motor which is horizontally installed on the linear motion sub-unit, and a second brushless DC motor which is vertically installed on the first brushless DC motor; and the first brushless DC motor and the second brushless DC motor are electrically connected with the main control unit.

15. The portable oropharyngeal swab smart collection device according to claim 14, wherein a sensing unit for acquiring a corresponding physical parameter is also arranged on the collection device body; the sensing unit is in communication connection with the visual display unit; the visual display unit is used for displaying the physical parameter transmitted by the sensing unit; and the sensing unit is electrically connected with the main control unit.

16. The portable oropharyngeal swab smart collection device according to claim 11, wherein a first emergency stop unit which is electrically connected with the main control unit is also arranged on the collection device body; and the remote control module further comprises a second emergency stop unit which is in communication connection with the main control unit.

17. The portable oropharyngeal swab smart collection device according to claim 11, wherein a detachable disposable bite block unit is arranged on the collection device body.

18. The portable oropharyngeal swab smart collection device according to claim 11, wherein an expansion interface with which external equipment is connected is arranged on the collection device body.

19. The portable oropharyngeal swab smart collection device according to claim 11, wherein an alarm unit is also arranged on the collection device body; and the alarm unit is in communication connection with the main control unit.

20. The portable oropharyngeal swab smart collection device according to claim 11, wherein further comprising a remote voice unit, which is used for a communication exchange between an operator and a patient.
